(19) United States
(12) Patent Application Publication
(10) Pub. No.: US 2014/0248696 A1
(43) Pub. Date: Sep. 4, 2014

(54) METHODS OF MAINTAINING, EXPANDING, AND DIFFRENTIATING NEURONAL SUBTYPE SPECIFIC PROGENITORS

(71) Applicant: Wisconsin Alumni Research Foundation, Madison, WI (US)

(72) Inventors: Su-Chun Zhang, Waunakee, WI (US); Zhong-wei Du, Madison, WI (US); Jianfeng Lu, Madison, WI (US)

(73) Assignee: Wisconsin Alumni Research Foundation, Madison, WI (US)

(21) Appl. No.: 14/194,130

(22) Filed: Feb. 28, 2014

Related U.S. Application Data
(60) Provisional application No. 61/771,572, filed on Mar. 1, 2013.

(51) Int. Cl. C12N 5/0797

(52) U.S. Cl. CPC C12N 5/0623
USPC 435/366; 435/377

(57) ABSTRACT

Methods for expanding proliferating populations of neuronal subtype-specific progenitors are provided herein. In particular, the present invention provides methods for maintaining the unique gene profile and differentiation potential of neuronal subtype-specific progenitors, such as motor neuron progenitors and hindbrain serotonergic neural progenitors.

### CROSS-REFERENCE TO RELATED APPLICATIONS

This application claims the benefit of U.S. Provisional Patent Application No. 61/771,572, filed Mar. 1, 2013, which is incorporated herein by reference as if set forth in its entirety.

### STATEMENT REGARDING FEDERALLY SPONSORED RESEARCH OR DEVELOPMENT

This invention was made with government support under grant number NS074189 awarded by the National Institutes of Health. The government has certain rights in the invention.

### FIELD OF THE INVENTION

The present invention relates to methods of expanding the population of neuronal subtype specific progenitors differentiated from human pluripotent stem cells, such as spinal motor neuron progenitors and hindbrain serotonergic neuron progenitors. In particular, the present invention relates to methods of maintaining the regional identity and differentiation potential of neuronal subtype specific progenitors during expansion.

### BACKGROUND OF THE INVENTION

The mammalian central nervous system is a complex neuronal network consisting of a diverse array of cellular subtypes generated in a precise spatial and temporal pattern throughout development. Each neuronal subtype within a particular region of the brain and spinal cord carries a unique set of neurotransmitters and establishes connections with its own targets. It is the diversity in molecular and morphological characteristics of neurons which underlies neural circuit formation.

Extrinsic signals provide neuronal progenitors in the forming neural tube with positional identity, such that distinct types of neuronal progenitors express a unique combination of transcription factors. This transcriptional code determines neural progenitor identity. As progenitors differentiate, they generate distinct neuronal subtypes that are also characterized by transcriptional codes and secretion of specific transmitters. For example, motor neurons (MNs) are a highly specialized class of neurons that reside in the spinal cord and project axons in organized and discrete patterns to muscles to control their activity. Motor neurons secrete the transmitter acetylcholine, express transcription factors including MNX1 (also known as HB9), ISL1, and LHX3, and are derived from motor neuron progenitors which express the basic helix-Loop-helix (bHLH) transcription factor OLIG2. During neurogenesis, OLIG2 is expressed by pMN cells and is required for the generation of MNs, while the homeodomain protein NKX2.2 is expressed in p3 progenitors and induces V3 neurons. Dessaud et al., Development 135:2489-2503 (2008). The most prominent MN diseases are spinal muscular atrophy (SMA) and amyotrophic lateral sclerosis (ALS), in which MNs perish in the disease. For review, see Kanning et al., Annu. Rev. Neurosci. 33:409-410 (2010). Similarly, hindbrain serotonin neuronal progenitors express NKX2.2 together with GATA2 but not OLIG2 or PHOX2b and generate serotonin-secreting neurons that project to the entire brain and spinal cord. Numerous psychiatric disorders involve dysfunctional serotonin neurons. For review, see Gordis & Rohrer, Nat. Rev. Neurosci. 3(7):531-541 (2002); Kiyasova & Gaspar, Eur. J. Neurosci. 34(10):1553-1562 (2011).

Neural progenitor cells have been expanded in culture in the presence of mitogens such as epidermal growth factor (EGF) and/or fibroblast growth factor 2 (FGF2). For review, see Weiss et al., Trends Neurosci. 19:387-393 (1996). Neural progenitors expanded under such conditions exhibit diminished potential for generating neurons over glial cells. See Temple, Nature 414:112-117 (2001). This trend is in general agreement with the shift from neurogenesis to gliogenesis observed during normal development. Embryonic ventral mesencephalic progenitors, which produce robust dopaminergic neurons at the time of isolation, lose their dopaminergic potential shortly after expansion in the presence of FGF2. See Studer et al., Nat. Neurosci. 1:290-295 (1998). Similarly, human embryonic stem cell (ESC)-derived neural progenitors retain their positional identity, as determined by homeodomain transcription factor expression, and a high degree of neurogenic potential even after months of expansion. See Zhang et al., J. Hematother. Stem Cell Res. 12:625-634 (2003). The potential to produce large projection neurons such as midbrain dopamine neurons, spinal cord motor neurons, and hindbrain serotonergic neurons, however, fades within two to four passages and is replaced by other neuronal populations. This phenomenon creates a barrier for producing consistent populations of neuronal progenitors with predictable differentiation potential and functional properties. Accordingly, there remains a need for compositions and methods for expanding neuronal progenitors while maintaining the differentiation potential of the progenitors to yield the predicted array of diverse neuronal subtypes.

### SUMMARY OF THE INVENTION

In one aspect, the present invention provides a method for maintaining a population of neuronal subtype-specific progenitors. The method can comprise culturing neuronal subtype-specific progenitors in a culture medium comprising a Wnt signaling pathway agonist, an inhibitor of the Bone Morphogenetic Protein (BMP) signaling pathway, an inhibitor of the transforming growth factor beta (TGFβ) signaling pathway, and a Notch signaling pathway agonist whereby expression of a neuronal subtype-specific progenitor gene expression profile is maintained in the neuronal subtype-specific progenitors. The neuronal subtype-specific progenitors can have a gene expression profile comprising expression of at least one of SOX1, SOX2, NESTIN, N-Cadherin, and Ki67. The neuronal subtype-specific progenitors can be spinal neural progenitors having a gene expression profile further comprising expression of at least one of HOXA5 and HOXB8, and substantially no expression of midbrain, hindbrain, or forebrain markers. The spinal neural progenitors can be OLIG2⁺ spinal motor neuron progenitors.

The neuronal subtype-specific progenitors can be hindbrain neural progenitors having a gene expression profile further comprising expression of at least one of GBX2, KROX20, HOXA1-4, and HOXB1-4, and substantially no expression of forebrain, spinal cord, or midbrain markers. The hindbrain neural progenitors can be NKX2.2⁺ hindbrain serotonergic neural progenitors.

In some cases, the neuronal subtype-specific progenitors are midbrain neural progenitors having a gene expression profile further comprising expression of at least one of EN1 and EN2, and substantially no expression of forebrain, spinal cord, or hindbrain markers. The midbrain neural progenitors can be LMX1A⁺ midbrain dopaminergic neuron progenitors.

The neuronal subtype-specific progenitors can be forebrain neural progenitors having a gene expression profile further comprising expression of at least one of FOXG1 and OTX2, and substantially no expression of midbrain, spinal cord, or hindbrain markers. The forebrain neural progenitors can be NKX-2.1⁺ forebrain GABAergic neuron progenitors.

The Wnt signaling pathway agonist can be a GSK3 inhibitor selected from the group consisting of CHIR99021 and 6-bromo-iridium-3′-oxime. The BMP signaling pathway inhibitor can be selected from the group consisting of DMH-1, Dorsomorphin, and LDN-193189. The Notch signaling pathway agonist can be a histone deacetylase (HDAC) inhibitor selected from the group consisting of valproic acid (VPA), suberoyl bis-hydroxamic acid (SBHA), and sodium butyrate. The TGFIβ signaling pathway inhibitor can be selected from the group consisting of SB431542, SB505124, and A83-01. The culture medium can comprise CHIR99021, DMH-1, SB431542, and VPA. The culture medium can comprise between about 1 μM-3 μM CHIR99021; about 1 μM-5 μM DMH-1; about 1 μM-5 μM SB431542; and about 0.2-μM-2 μM VPA.

The neuronal subtype specific progenitors can be OLIG2⁺ spinal motor neuron progenitors, where the culture medium comprises CHIR99021, DMH-1, SB431542, VPA, a SHH pathway agonist, and a RA pathway agonist. The SHH pathway agonist can be selected from the group consisting of purmorphamine and SAG (Smoothened Agonist). The RA pathway agonist can be retinoic acid. The culture medium can comprise between about 1 μM to 3 μM CHIR99021; about 1 μM to 5 μM DMH-1; about 1 μM to 5 μM SB431542; about 0.2 μM -2 μM VPA; and about 0.1 μM to 1 μM purmorphamine; about 0.01 μM to 1 μM RA. The OLIG2⁺ spinal motor neuron progenitors can be maintained in a culture substantially free of MNX1⁺ post-mitotic motor neurons for at least 5 weeks. The OLIG2⁺ spinal motor neuron progenitors can be maintained in a culture substantially free of MNX1⁺ post-mitotic motor neurons for at least 10 weeks.

The neuronal subtype specific progenitors can be NKX2.2⁺ hindbrain serotonergic neural progenitors, where the culture medium comprises CHIR99021, DMH-1, SB431542, VPA, and purmorphamine. The culture medium can comprise about 1 μM to 3 μM CHIR99021; about 1 μM to 5 μM DMH-1; about 1 μM to 5 μM SB431542; about 0.2 μM-2 μM VPA; and about 0.1 μM to 1 μM purmorphamine. The NKX2.2⁺ hindbrain serotonergic neural progenitors can be maintained substantially free from differentiation for at least 5 weeks. The NKX2.2⁺ hindbrain serotonergic neural progenitors can be maintained substantially free from differentiation for at least 10 weeks.

In some cases, neuronal subtype specific progenitors are obtained from pluripotent stem cells. The pluripotent stem cells can be human pluripotent stem cells. The human pluripotent stem cells can be human embryonic stem cells or human induced pluripotent stem cells. The neuronal subtype specific progenitors can be obtained from a human embryo.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

FIG. 1 is a flow chart depicting differentiation of spinal motor neuron progenitors and hindbrain serotonergic neuron progenitors from pluripotent stem cells and expansion of these neuronal progenitors under specified conditions. Abbreviations: PSC (pluripotent stem cell); NE (neuroepithelial progenitor); MNP (motor neuron progenitor); SNP (serotonergic neural progenitor); RA (retinoic acid); MN (motor neuron).

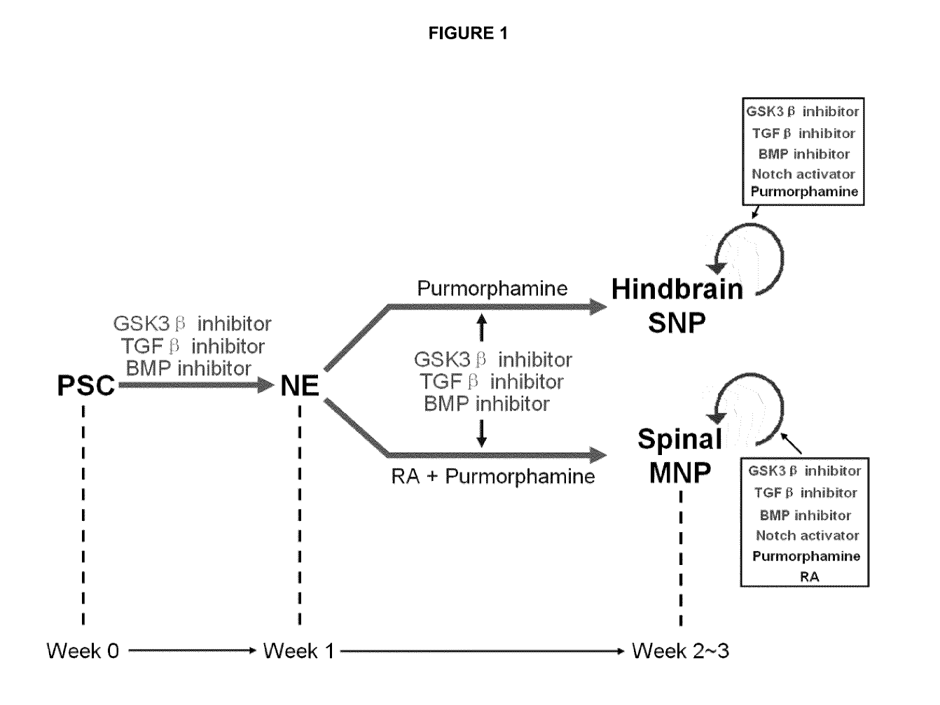

FIG. 2 is a flow chart depicting an exemplary protocol for differentiating mature motor neurons from an expanded population of spinal motor neuron progenitors.

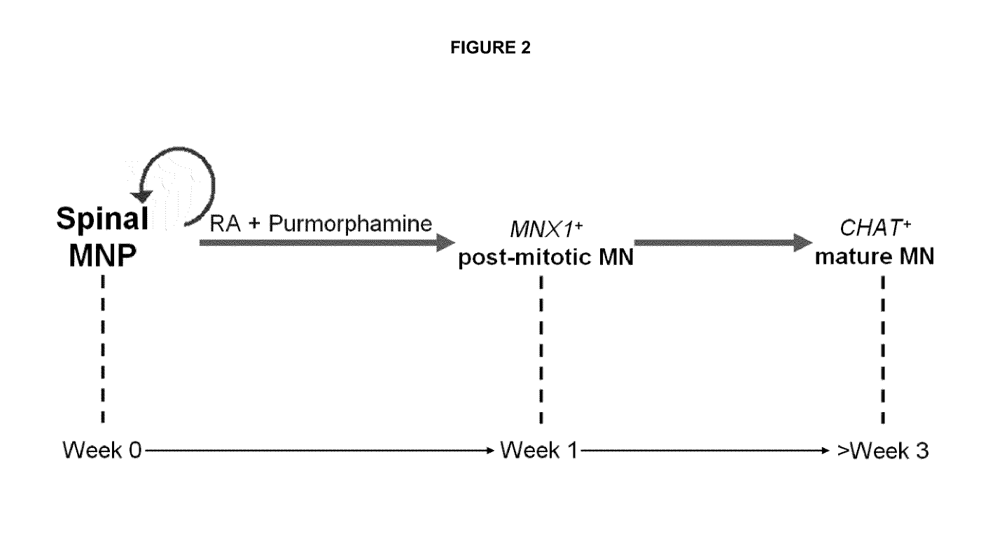

### DETAILED DESCRIPTION OF THE INVENTION

The present invention is based, at least in part, on the inventors' discovery that a defined cocktail of small molecules or chemical compounds could be used to maintain the proliferation of neuronal subtype-specific progenitor cells, such as spinal motor neuron progenitors and hindbrain serotonergic neuron progenitors differentiated from human pluripotent stem cells. The inventors further discovered that certain culture conditions could maintain in vitro cultured neuronal subtype-specific progenitor cells in their progenitor state with substantially no loss of differentiation potential. Upon providing a differentiation condition to the maintained, expanded progenitors, the Inventors induced differentiation of the progenitors into, for example, mature motor neurons and serotonergic neurons.

Methods of Generating and Maintaining Neuronal Subtype-Specific Progenitors

In one aspect, therefore, the present invention is directed to methods for generating and maintaining a population of neuronal subtype specific progenitors. Neuronal subtypes-specific progenitors can include, without limitation, forebrain neural progenitors, spinal neural progenitors, hindbrain neural progenitors, and midbrain neuron progenitors. The phenotype of a neuronal subtype specific progenitor is specified by the expression of unique combination of transcription factors in rostral-caudal and dorsal-ventral patterns. For example, forebrain neural progenitors can be NKX-2.1⁺ forebrain GABAergic neuron progenitors, and midbrain neural progenitors can be TH (tyrosine hydroxylase)-expressing midbrain dopaminergic neuron progenitors.

A method for generating a population of neuronal subtype-specific progenitors can include culturing neuroepithelial cells in a culture medium comprising a Wnt signaling pathway agonist, an inhibitor of the bone morphogenetic protein (BMP) signaling pathway, an inhibitor of the transforming growth factor beta (TGFβ) signaling pathway, and Notch signaling pathway agonist, and at least one of retinoic acid (RA) or a sonic hedgehog (SHH) pathway agonist, where the cells are cultured for a time sufficient to induce expression of a neuronal subtype-specific progenitor gene expression profile. A neuronal subtype-specific gene expression profile will include expression of at least one of SOX1, SOX2, NESTIN, N-Cadherin, and Ki67. In the case of hindbrain neural progenitors, the gene expression profile can further include at least one of GBX2, KROX20, HOXA1-4, and HOXB1-4, but substantially no expression of forebrain, spinal cord, or midbrain markers. In an exemplary embodiment, a hindbrain neural progenitor is a NKX2.2⁺ hindbrain serotonergic neural progenitor. For midbrain neural progenitors, the gene expression profile can further include at least one of EN1, LMX1A, LMX1B, SIM1, and LIM1, but substantially no expression of forebrain, spinal cord, or hindbrain markers. In some cases, a midbrain neural progenitor is a midbrain dopaminergic neuron progenitor. For forebrain neural progenitors, the gene expression profile can further include at least one of FOXG1, OTX2, EMX1, NKX2.1, and SIX3, but substantially no expression of midbrain, spinal cord, or hindbrain markers. In some cases, the forebrain neural progenitor is a NKX-2.1⁺ forebrain GABAergic neuron progenitor. For a spinal neural progenitor, the gene expression profile can further include at least one of HOXB6 and HOXB8, but substantially no expression of midbrain, hindbrain, or forebrain markers. In some cases, the spinal neural progenitor is a OLIG2⁺ spinal motor neuron progenitor.

In some cases, a method for generating neuronal subtype-specific progenitors can further comprise culturing pluripotent stem cells in a culture medium for a time sufficient to induce differentiation of the pluripotent stem cells into neuroepithelial cells. The culture medium can comprise (i) a Wnt signaling pathway agonist, (ii) an inhibitor of the BMP signaling pathway, and (iii) an inhibitor of the TGFβ signaling pathway. Pluripotent stem cells that can be used include human pluripotent stem cells such as human embryonic stem cells and human induced pluripotent stem cells.

Methods of maintaining a population of neuronal subtype specific progenitors derived from pluripotent stem cells can comprise culturing neuronal subtype specific progenitors in a culture medium comprising a Wnt signaling pathway agonist, an inhibitor of the bone morphogenetic protein (BMP) signaling pathway, an inhibitor of the transforming growth factor beta (TGFβ) signaling pathway, and a Notch signaling pathway agonist. In some cases, such a culture medium is called a maintenance culture medium. By “maintaining” a population of neuronal subtype-specific progenitors, we mean maintenance of a phenotype of a unique gene expression profile (e.g., profile of transcription factors expressed in a given cell type) characteristic of a given neuronal subtype specific progenitor. As used herein, the term “maintaining” refers to maintenance of such a phenotype (e.g., cell morphology, gene expression profile) characteristic of a given neuronal subtype specific progenitor for at least 5 passages or at least 5 weeks, preferably at least 8 passages or at least 8 weeks, and most preferably at least 10 passages or at least 10 weeks.

A culture medium comprising small molecule agonists of each of the Wnt and Notch signaling pathways, and small molecule inhibitors of the transforming growth factor beta (TGFβ) and BMP pathways are required for maintaining the proliferation and self-renewal of neuronal progenitors generally. However, other small molecules or patterning factors are additionally required for maintaining the unique gene expression profile characteristic of a neuronal subtype specific progenitor. For example, a Sonic Hedgehog (SHH) signaling pathway agonist (e.g., purmorphamine) and a retinoic acid (RA) signaling pathway agonist are additionally required to maintain expression of the transcription factor OLIG2 in motor neuron progenitors and to maintain motor neuron progenitor identity and differentiation capacity. Similarly, a SHH signaling pathway agonist is additionally required to maintain expression of the transcription factor NKX2.2 in hindbrain serotonergic neuron progenitors and to maintain hindbrain serotonergic neuron progenitor identity and differentiation capacity.

In an exemplary embodiment, a culture medium for maintaining a population of any type of other neuronal subtype specific progenitors according to a method provided herein comprises RA, purmorphamine, the GSK3 inhibitor CHIR99021, the BMP signaling inhibitor DMH-1, and the TGFIβ signaling inhibitor SB431542. In some cases, the culture medium further comprises between about 0.1 μM to 1.0 μM RA, and between about 0.1 μM to 1.0 μM purmorphamine.

In other cases, maintaining neuronal progenitors according to a method provided herein can include providing the cells with a culture medium comprising an agonist of Notch signaling such as, for example, VPA (Valproic acid). VPA is available from several commercial chemical compound vendors (e.g., Tocris Bioscience, Sigma-Aldrich). VPA is an HDAC inhibitor which can indirectly activate Notch signaling. Stockhausen et al., Br. J. Cancer. 92(4):751-759 (2005). Other small molecule inhibitors of HDAC which can be used to activate Notch signaling include, for example, suberoyl bis-hydroxamic acid (SBHA) and sodium butyrate. Accordingly, a culture medium appropriate for use in a method for maintaining neuroepithelial cells can comprise CHIR99021, DMH-1, SB431542, and at least one of valproic acid (VPA), a SHH pathway agonist, and RA (or RA pathway agonist). In some cases, the culture medium can comprise between about 1 μM-3 μM CHIR99021, between about 1 μM-5 μM DMH-1, between about 1 μM-5 μM SB431542, and at least one of between about 0.2 μM-2 μM VPA, between about 0.2μM-2 μM RA, and between about 0.2 μM-2 μM purmorphamine.

Any appropriate culture method can be used to practice a method provided herein. In an exemplary embodiment, adherent culture methods can be used. Adherent culture (or “colony culture”) allows direct visualization of neural differentiation, including the formation of neural tube-like rosettes during neuroepithelial induction and the migration of neuroepithelial cells. Adherent/colony culture permits ready removal of non-neural colonies and promotes subsequent neural differentiation. In some cases, suspension culture can be used for initially separating pluripotent cells from mouse embryonic fibroblast (MEF) feeder cells or for purifying neuroepithelial cells.

Methods of Generating and Maintaining Motor Neuron Progenitors

In another aspect, the present invention is directed to methods for generating motor neuron progenitors and methods for maintaining an expanded population of motor neuron progenitors. As used herein, the term “motor neuron progenitor” refers to a progenitor or precursor cell which will mature, or is capable of maturing, into a motor neuron.

To generate motor neuron progenitors, a first step in the method can be to generate a population of neuroepithelial cells. Neuroepithelial cells are also known as neural stem cells, and the terms “neuroepithelial cell” and “ neural stem cell” are used interchangeably throughout. A method for generating a population of motor neuron progenitors can comprise culturing human pluripotent stem cells in a culture medium comprising a Wnt signaling pathway agonist, an inhibitor of the BMP signaling pathway, and an inhibitor of the TGFβ signaling pathway for a time sufficient to induce differentiation of pluripotent stem cells into neuroepithelial cells. Pluripotent stem cells useful for the methods provided herein include human embryonic stem cells (hESCs) and human induced pluripotent stem cells (hIPS cells).

In some cases, a culture medium appropriate for generating a population of neuroepithelial cells can comprise a plurality of small molecules or other chemical compounds which promote the differentiation of pluripotent stem cells into neuroepithelial cells. In some cases, such a culture medium is called a differentiation culture medium. The plurality of small molecules or chemical compounds can include an agonist of the canonical Wnt signaling pathway, an inhibitor of the BMP signaling pathway, and an inhibitor of Activin/Nodal/TGFβ signaling. For example, a method for generating a population of neuroepithelial cells can include providing pluripotent stem cells with a culture medium comprising CHIR99021, a GSK3 inhibitor. By inhibiting GSK3, CHIR99021 activates the canonical Wnt signaling pathway. CHIR99021 has been reported to inhibit the differentiation of mouse and human embryonic stem cells (ESCs) through Wnt signaling. For review, see Wray and Hartmann, Trends in Cell Biology 22:159-168 (2012). Another GSK3 inhibitor which can be used is, for example, the Wnt/β-catenin signaling agonist 6-bromo-iridium-3′-oxime (“B10”). See Meijer et al., Chem. Biol. 10(12):1255-66 (2003). GSK3 inhibitors such as those described herein are available from commercial vendors of chemical compounds (e.g., Selleckchem, Tocris Bioscience).

In some cases, an inhibitor of BMP signaling is DMH-1, which blocks BMP signaling by inhibiting Activin receptor-like kinase (ALK2). Other small molecule inhibitors of Activin receptor-like kinases which can be used to block BMP signaling include, for example, Dorsomorphin and LDN-193189. Both compounds affect Smad-dependent and Smad-independent BMP signaling triggered by BMP2, BMP6, or GDF5. Boergermann et al., Int. J. Biochem. Cell Biol. 42(11):1802-7 (2010).

In some cases, an inhibitor of Activin/Nodal/TGFβ signaling is SB431542, which inhibits Activin receptor-like kinases 4, 5, and 7 (ALK4, ALK5, and ALK7). SB431542 can be purchased from any one of several commercial chemical compound vendors (e.g., Tocris Bioscience, Sigma-Aldrich). By inhibiting Activin receptor-like kinases 4, 5, and 7, SB431542 inhibits Activin/Nodal/TGFβ signaling. Other small molecule inhibitors of Activin receptor-like kinase 5 (ALK5) (also known as transforming growth factor-α type I receptor kinase) such as SB505124 and A83-01 can be used to inhibit Activin/Nodal/TGFβ signaling.

In an exemplary embodiment, a culture medium for use according to a method provided herein comprises the GSK3 inhibitor CHIR99021, the BMP signaling inhibitor DMH-1, and the TGFβ signaling inhibitor SB431542. In some cases, the culture medium can comprise between about 1 μM-3 μM CHIR99021, between about 1 μM-5 μM DMH-1, and between about 1 μM-5 μM SB431542.

In some cases, a culture medium for use according to a method provided herein comprises a basal culture medium supplemented with small molecules or chemical compounds such as those described herein. For example, a culture medium can be Neurobasal® culture medium (Life Technologies. In some cases, a culture medium comprises DMEM/F12, Neurobasal medium at 1:1, 1× N2 neural supplement (N-2 Supplement; Gibco), 1× B27 neural supplement (B-27 Supplement; Gibco), and 1 mM ascorbic acid.

A method for generating motor neuron progenitors can further comprise inducing neuroepithelial cells to differentiate into spinal motor neuron progenitors. In some cases, the method comprises culturing neuroepithelial cells in a culture medium comprising a Wnt signaling pathway agonist, an inhibitor of the BMP signaling pathway, an inhibitor of the TGFβ signaling pathway, a sonic hedgehog (SHH) signaling agonist, and a RA signaling agonist for a time sufficient to induce expression of a motor neuron progenitor marker (OLIG2).

In some cases, generating motor neuron progenitors according to a method provided herein can include providing neuroepithelial cells (e.g., stem cell-derived NE cells) with a culture medium comprising a SHH signaling pathway agonist such as, for example, purmorphamine. Purmorphamine is available from several commercial chemical compound vendors (e.g., Tocris Bioscience, Stemgent). Purmorphamine activates SHH signaling by directly targeting Smoothened (“Smo”), a critical component of the SHH signaling pathway. Sinha et al., Nature Chem. Biol. 2:29-30 (2006). Other small molecule agonists of Smo which can be used to activate SHH signaling include, for example, SAG (“Smoothened Agonist”). The hedgehog pathway agonist SAG is a cell-permeable chlorobenzothiophene compound that modulates the coupling of Smo with its downstream effector by interacting with the Smo heptahelical domain. SHH acts in a graded manner to establish different neural progenitor cell populations. See Briscoe et al., Semin. Cell Dev. Biol. 10(3):353-62 (1999).

In some cases, generating motor neuron progenitors according to a method provided herein can include providing cells with a culture medium comprising a RA signaling agonist such as RA (retinoic acid). RA is available from several commercial chemical compound vendors (e.g., Tocris Bioscience, Sigma-Aldrich). RA activates RA signaling by binding nuclear hormone receptors retinoic acid receptors (RARs), which is required for specification of motor neuron progenitors. See Novitch et al., Neuron 40(1):81-95 (2003).

In an exemplary embodiment, a culture medium for generating motor neuron progenitors according to a method provided herein comprises Wnt signaling agonist CHIR99021, BMP signaling inhibitor DMH-1, TGFIβ signaling inhibitor SB431542, SHH signaling agonist purmorphamine, and retinoic acid. In some cases, the culture medium comprises between about 1 μM-3 μM CHIR99021, between about 1 μM-5 μM DMH-1, between about 1 μM-5 μM SB431542, between about 0.2 μM-2 μM purmorphamine, and between about 0.1 μM-1.0 μM RA.

Cells cultured and differentiated according to a method provided herein can be identified as motor neuron progenitors on the basis of OLIG2⁺ expression. The bHLH transcription factor OLIG2 serves as a unique marker of MN progenitors. The transcriptional repressor function of OLIG2 is both necessary and sufficient to stimulate the expression of a number of downstream homeodomain transcription factors that provide MNs with their unique character. See Briscoe and Novitch, Philos. Trans. R. Soc. Lond. B. Biol. Sci. 363(1489):57-70 (2008); see also Shirasaki and Pfaff, Annu. Rev. Neurosci. 25:251-281 (2002).

In some cases, a method provided herein further includes a step of culturing OLIG2⁺ motor neuron progenitors in a MN progenitor differentiation culture medium for approximately one week to generate MNX1⁺ post-mitotic motor neurons. MNX1 (also known as Motor Neuron and Pancreas Homeobox 1 or H89) is homeobox gene expressed selectively by motor neurons in the developing vertebrate central nervous system (Arber et al., Neuron 23(4):659-74 (1999)). Alternatively, post-mitotic motor neurons can be marked by the expression of ISLET1/2. In some cases, a method provided herein further includes culturing OLIG2⁺ motor neuron progenitors in a MN progenitor differentiation culture medium for at least about two weeks (e.g., 2 weeks, 2.5 weeks, 3 weeks) to generate choline acetyltransferase-positive (ChAr) mature motor neurons. Choline acetyltransferase is an enzyme that catalyzes the synthesis of the transmitter acetylcholine for transmitting signals through the neuromuscular junctions and is expressed in somatic, cholinergic (acetylcholine-producing) motor neurons. Mature motor neurons also express VAChAT (vesicular acetylcholine transporter), a neurotransmitter transporter which is essential for storage of acetylcholine (ACh) in secretory organelles and for release of ACh.

In another aspect, the present invention is directed to methods for maintaining a population of motor neuron progenitors. As used herein, the term “maintaining” refers to maintenance of a phenotype (e.g., cell morphology, gene expression profile, differentiation potential) characteristic of a given progenitor for at least 5 weeks, preferably at least 8 weeks, and most preferably at least 10 weeks. For example, the present invention provides methods for maintaining OLIG2⁺ motor neuron progenitors in vitro for at least 5 weeks.

Methods of maintaining a population of motor neuron progenitors derived from pluripotent stem cells can comprise culturing motor neuron progenitors in a culture medium comprising a Wnt signaling pathway agonist, an inhibitor of the BMP signaling pathway, an inhibitor of the TGFβ signaling pathway, a Notch signaling pathway agonist, a SHH signaling pathway agonist, and a RA signaling pathway agonist. In some cases, such a culture medium is called a maintenance culture medium. Maintaining cells according to a method provided herein can include providing cells with a culture medium comprising an agonist of Notch signaling such as, for example, VPA (Valproic acid). VPA is available from several commercial chemical compound vendors (e.g., Tocris Bioscience, Sigma-Aldrich). VPA is a histone deacetylase (HDAC) inhibitor which can indirectly activate Notch signaling. Stockhausen et al., Br. J. Cancer. 92(4):751-759 (2005). Other small molecule inhibitors of HDAC which can be used to activate Notch signaling include, for example, suberoyl bis-hydroxamic acid (SBHA) and sodium butyrate. Accordingly, a culture medium appropriate for use in a method for maintaining motor neuron progenitors can comprise CHIR99021, DMH-1, SB431542, VPA, purmorphamine, and RA.

In an exemplary embodiment, methods for maintaining a population of motor neuron progenitors can comprise culturing motor neuron progenitors in a culture medium comprising between about 1 μM-3 μM CHIR99021; between about 1 μM-5 μM DMH-1; between about 1 μM-5 μM SB431542; between about 0.2 μM-2 μM VPA; between about 0.2 μM-2 μM purmorphamine; and between about 0.1 μM-1.0 μM RA. Under these conditions, motor neuron progenitors maintain long-term OLIG2⁺ expression without differentiating or switching into other neural progenitor subtypes such as NKX2.2⁺ V3 interneuron progenitors (p3 domain progenitors). The motor neuron progenitors can be maintained for at least 5 weeks (e.g., at least about 5 passages), yielding previously unobtainable numbers of MN progenitors (producing on the order of 10⁴ MN progenitors from a single MN progenitor cell).

Methods of Generating and Maintaining Hindbrain Serotonergic Neuron Progenitors

In a further aspect, the present invention is directed to methods for generating and maintaining a population of hindbrain serotonergic neuron progenitors. The terms “serotonergic neuron progenitor” and “serotoninergic neural progenitor” are used interchangeably throughout and refer to a progenitor or precursor cell which will mature into a neuron capable of serotonin neurotransmission.

Methods of generating a population of hindbrain serotonergic neuron progenitors can comprise culturing neuroepithelial cells in a culture medium comprising a Wnt signaling pathway agonist, an inhibitor of the BMP signaling pathway, and an inhibitor of the TGFIβ signaling pathway plus a SHH signaling pathway agonist for a time sufficient (e.g., about 1 week to about 2 weeks) to induce expression of a hindbrain marker. Hindbrain serotonergic neuron progenitors generated from human pluripotent stem cells according to a method provided herein can be defined based on their expression of hindbrain markers (e.g., GBX2, KROX20, HOXA1-4, HOXB1-4), but not forebrain markers (e.g., FOXG1, OTX2, EMX1, NKX2.1, SIX3), midbrain markers (e.g., EN1, LMX1A, LMX1B, SIM1, LIM1), or spinal cord markers (e.g., HOXB6, HOXB8) besides the neural progenitor markers (e.g., SOX1, SOX2, NESTIN, N-Cadherin, and Ki67).

In an exemplary embodiment, a culture medium for generating a population of hindbrain serotonergic neuron progenitors according to a method provided herein comprises Wnt signaling agonist CHIR99021, BMP signaling inhibitor DMH-1, TGFIβ signaling inhibitor SB431542, and SHH signaling pathway agonist purmorphamine. In some cases, the culture medium can comprise between about 1 μM-3 μM CHIR99021; between about 1 μM-5 μM DMH-1; between about 1 μM-5 μM SB431542; and between about 0.2 μM-2 μM purmorphamine.

Methods for maintaining a population of hindbrain serotonergic neuron progenitors can comprise culturing hindbrain serotonergic neuron progenitors in a maintenance medium comprising between about 1 μM-3 μM CHIR99021; between about 1 μM-5 μM DMH-1; between about 1 μM-5 μM SB431542; between about 0.2 μM-2 μM VPA, and between about 0.2 μM-2 μM purmorphamine. Under these conditions, serotonergic neuron progenitors maintain long-term NKX2.2⁺ expression without switching into other neural progenitor subtypes. The serotonergic neural progenitors can be maintained for at least 5 weeks or at least 5 passages.

Methods of Maintaining other Neuronal Subtype Specific Progenitors

In a further aspect, the present invention is directed to methods for maintaining a population of any other type of neuronal subtype specific progenitors, for example, forebrain GABAnergic neuron progenitors, or midbrain dopaminergic neuron progenitors. The phenotype of neuronal subtype specific progenitors is defined by a unique gene expression profile of regional markers and subtype specific markers, and the potential to differentiate into subtype specific mature neurons, For example, forebrain GABAnergic neuron progenitor is defined by expression of forebrain markers FOXG1, OTX2B and subtype specific marker NKX2.1, as well as its ability to differentiate into mature neuron secreting GABA neurotransmitter. Similarly, a midbrain dopaminergic neuron progenitor is marked by midbrain transcription factors EN1, EN2 and subtype specific transcription factor LMX1A, as well as its potential to differentiate into mature neuron secreting dopamine neurotransmitter. By “maintaining” a population of progenitors, we mean maintenance of a phenotype for at least 5 passages or at least 5 weeks, preferably at least 8 passages or at least 8 weeks, and most preferably at least 10 passages or at least 10 weeks.

Methods of maintaining a population of neuronal subtype specific progenitors can comprise culturing neuronal subtype specific progenitors in a culture medium comprising a Wnt signaling pathway agonist, an inhibitor of the BMP signaling pathway, an inhibitor of the TGFIβ signaling pathway, a Notch signaling pathway agonist.

The four small molecules, CHIR99021, DMH-1, SB431542, and VPA, in the core maintaining medium are required for maintaining the proliferation and phenotype of progenitors. However, other small molecules or patterning factors may be required for maintaining the unique subtype specific progenitors. For example, a SHH signaling pathway agonist and a RA signaling pathway agonist are required in maintaining OLIG2 transcription factor expression in spinal motor neuron progenitors; a SHH signaling pathway agonist is required in maintaining NKX2.2 transcription factor expression in hindbrain serotonergic neuron progenitors.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1

### Efficient Generation of MN Progenitors From hESCs in 2 Weeks

To induce the specification of neuroepithelial cells from human pluripotent cells, the dual Nodal/BMP inhibition approach was applied for human embryonic stem cells in a monolayer culture. See, for review, Chambers et al., Nature Biotech. 27:275-280 (2009). The small molecule SB431542 represses Nodal/Activin signaling by selectively inhibiting Activin receptor-like kinase ALK4/5/7. The small molecule DMH-1 represses BMP signaling by selectively inhibiting the BMP receptor kinase ALK2. Human embryonic stem cells (hESCs) were treated with 2 μM DMH-1 and 2 μM SB431542 for 1 week. Treated hESCs were then induced to differentiate into populations comprising about 85% SOX1⁺ neuroepithelial cells but also comprising other cell lineages due to spontaneous ESC differentiation, since the dual Nodal/BMP inhibitors SB431542 and DMH-1 are unable to prevent all spontaneous differentiation into other cell lineages, especially when ESC colonies are small. As described herein, a small molecule that inhibits glycogen synthase kinase-3 (CHIR99021) maintain the ESC state during culturing.

To further improve neural specification, a small molecule that inhibits glycogen synthase kinase-3 (CHIR99021) was applied in combination with DMH-1 and SB431542. GSK3 negatively regulates WNT signaling, and WNT signaling promotes the self-renewal of ESCs and neural progenitors. When exposed to these three molecules for about 6 days, hESCs not only generated more pure populations of SOX1⁺ neuroepithelial cells (e.g., at least 95% of cells in the total population were SOX1⁺ neuroepithelial cells), but also generated 2.5-fold more neuroepithelial cells. However, CDS (CHIR99021, DMH-1, and SB431542) treatment-derived neuroepithelial cells showed caudal identity as demonstrated by staining for HOXA2. By contrast, DS (DMH-1 and SB431542) treatment-derived neuroepithelial cells showed rostral identity as demonstrated by staining for OTX2.

The efficiency of motor neuron generation from these two populations of neuroepithelial cells was then compared. After treatment with 0.1 μM Retinoic Acid (RA) and 1 μM purmorphamine (a small molecule for activating SHH signaling) for another 6 days, more Than90% OLIG2⁺ MN progenitors were induced from CDS treatment-derived neuroepithelial cells, but only 60% from DS treatment-derived neuroepithelial cells. These data suggest an efficient approach for inducing MN progenitors from pluripotent stem cells by contacting the stem cells with a three-molecule cocktail of CDS (CHIR99021, DMH-1, and SB431542) or another cocktail of compounds affecting the Wnt pathway, the BMP pathway, and the Activin/Nodal signaling pathway, respectively, as described herein.

Since the pMN domain is patterned by a gradient of SHH signaling, the efficiency of MN generation upon exposure to different concentrations of purmorphamine was examined. It was observed that 0.5 μM purmorphamine induced a similarly pure population of OLIG2⁺ MN progenitors as 1 μM (approximately 90% OLIG2⁺ MN progenitors), but induced few NKX2.2⁺ p3 progenitors (V3 interneuron progenitors). Concentrations of less than 0.5 μM purmorphamine induced the fewest number of OLIG2⁺ MN progenitors.

### Example 2

### Long-term Expansion of OLIG2⁺ MN Progenitors

Next, we examined whether OLIG2⁺ MN precursors could be maintained as a continuously dividing population. OLIG2⁺ MN precursors obtained from the 2-week differentiation were split and cultured under CDS conditions (i.e., in the presence of the 3-molecule CDS cocktail) plus 0.1 μM RA and 0.5 μM purmorphamine. However, the cells gradually lost their dividing potential and became post-mitotic MNs as determined by staining for MNX1, which suggested that RA induces the exit of cell cycle and promotes neurogenesis. Withdrawing RA from the culture was attempted. In the presence of the CDS cocktail plus 0.5 μM purmorphamine, the cells expanded but the neural precursors gradually lost OLIG2 expression and increased NKX2.2 expression, which suggested that purmorphamine alone cannot maintain MN precursors. Instead, the cells switch into p3 domain precursors. Next, motor neuron progenitors were cultured with the CDS cocktail plus RA , purmorphamine, and plus 0.5 μM VPA. VPA can activate Notch signaling pathway, which blocks the neurogenesis induced by RA. This condition can maintain a substantially pure population of OLIG2⁺ MN progenitors without inducing MNX1⁺ MNs (e.g., a population substantially devoid of MNX1⁺ motor neurons) and switch into p3 domain precursors.

Among the four small molecules of the “maintaining” culture medium, CHIR99021 was the core factor for the expansion of MN progenitors since withdrawal of CHIR99021 resulted in a significant loss of dividing potential. DMH-1 and SB431542 cooperated with CHIR99021 to obtain the maximal proliferation. VPA repressed the neurogenesis by blocking the expression of neurogenic transcription factors Ngn2 and Ngn1. RA and purmorphamine are required to maintain the expression of MN progenitor marker OLIG2, which means maintaining the identity and differentiation potential of MN progenitors. Under these conditions, OLIG2⁺ MN progenitors can be maintained and expanded in culture for at least 5 weeks (e.g., at least 5 passages), yielding previously unobtainable numbers of MN progenitors (on the order of producing 10⁴ MN progenitors from a single MN progenitor cell). It was also observed that OLIG2⁺ MN precursors can be frozen in liquid nitrogen. When thawed and cultured in MN differentiation medium, OLIG2⁺ MN progenitors differentiated into MNX1⁺ post-mitotic motor neurons in 1 week and further into CHAT mature motor neurons in 2-3 weeks.

### Example 3

### Differentiating and Maintaining Hindbrain Serotonergic Neural Progenitors

Human embryonic stem cells or induced pluripotent stem cells were seeded onto laminin-coated plates and cultured in human ESC medium for 1 day. On the following day, the culture medium was changed to Neurobasal culture medium comprising 2 μM SB431542, 2 μM DMH1, and 1.0-3.0 μM CHIR99021 for one week. Neural progenitors having hindbrain identity were generated from human pluripotent stem cells. The hindbrain neural progenitors were defined by their expression of hindbrain makers (e.g., GBX2, KROX20, HOXA1-4, HOXB1-4), but not forebrain markers (e.g., FOXG1, OTX2, EMX1, NKX2.1, SIX3), midbrain markers (e.g., EN1, LMX1A, LMX1B, SIM1, LIM1), or spinal cord markers (e.g., HOXB6, HOXB8) besides the neural progenitor markers (e.g., SOX1, SOX2, NESTIN, N-Cadherin, and Ki67).

To differentiate neural progenitors toward the serotonergic neural cell fate, hindbrain neural progenitors were cultured in a medium comprising 1000 ng/mL C25II Sonic Hedgehog (SHH) or 1 μM purmorphamine for one week. The resultant cells became ventral hindbrain progenitors expressing hindbrain makers (e.g., GBX2, KROX20, HOXA1-4, HOXB1-4), but not forebrain markers (e.g., FOXG1, OTX2, EMX1, NKX2.1, SIX3). The resultant cells also expressed ventral hindbrain markers OLIG2, NKX6.1, and NKX2.2. The percentage of NKX2.2⁺ cells was as high as 91% of total cells assessed using a FACS assay. These ventral hindbrain neural progenitors could be maintained in a maintenance culture medium comprising 3.0 μM CHIR99021 and 1000 ng/mL C25II Sonic Hedgehog (SHH) or 1 μM purmorphamine for at least 5 passages. The ventral hindbrain neural progenitors were seeded onto polyornithine-coated coverslips, laminin-coated coverslips, or laminin-coated plates for further differentiation in a neural differentiation medium comprising 2.5 μM DAPT (a γ-secretase inhibitor and indirect inhibitor of Notch, a γ-secretase substrate) to enhance maturation.

### Example 4

### Materials and Experimental Procedures

Human ESC lines H9 and H1 (WiCell Institute, NIH Code 0062 and 0043, passages 18-35) and human iPSC lines (iSMA13 and iSMA23) were cultured on irradiated mouse embryonic fibroblasts (MEFs) as described in the standard hESC protocol available at wicell.org on the World Wide Web.

Retinoic acid, purmorphamine, and SHH stock solutions for addition to a culture medium described herein can be prepared as described by Hu and Zhang (Methods Mol. Biol. 636:123-137, 2010).

Generation of OLIG2⁺ MN progenitors using a monolayer differentiation method: After treating with 1 mg/ml Dispase, hPSCs were split 1:6 on irradiated MEFs. On the following day, the culture medium was replaced with neural medium (DMEM/F12, Neurobasal® culture medium (Life Technologies) at 1:1, 1× N2 neural supplement, 1× B27 neural supplement, 1 mM ascorbic acid). 3 μM CHIR99021, 2 μM DMH-1, and 2 μM SB431542 were added in fresh medium. The culture medium was changed daily. Human PSCs maintained under these conditions, without MEFs, for one week were induced into neuroepithelial cells. When treated with 1 mg/ml Dispase, neuroepithelial cells were split at 1:6 on irradiated MEF with the same medium described above. 0.1 μM RA and 0.5 μM purmorphamine were added in combination with CHIR99021, DMH-1, and SB431542. The medium was changed daily. Neuroepithelial cells maintained under these conditions for one week differentiated into OLIG2⁺ MN progenitors.

Generation of OLIG2⁺ MN progenitors using a suspension differentiation method: After treating with 1 mg/ml Dispase, hPSCs were lifted and cultured as cell aggregates in suspension in hESC medium (DMEM/F12 medium +20% KnockOut™ Serum Replacement (Gibco) supplement, 1× NMAA, 1× glutamax) for four days. On day 4, the hESC medium was replaced with neural medium (DMEM/F12, Neurobasal® culture medium (Life Technologies) at 1:1, 1× N2 neural supplement, 1× B27 neural supplement, 1 mM ascorbic acid). After culturing for another two days, the cell aggregates were attached on the culture plate. The neural medium was changed every other day. After culturing under these conditions for one week, hPSCs were induced into neuroepithelial cells. After treating with 1 mg/ml Dispase, neuroepithelial cells were lifted again and cultured as neurospheres in suspension. 0.1 μM RA and 0.5 μM purmorphamine were added in neural medium. The medium was changed every other day. Neuroepithelial cells maintained under these conditions for ten days differentiated into OLIG2⁺ MN progenitors.

Maintenance of OLIG2⁺ MN progenitors: OLIG2⁺ MN progenitors can be frozen in regular freezing medium (DMEM/F12, 10% fetal bovine serum, 10% DMSO). To passage, MN progenitors were treated with 1 mg/ml Dispase and split 1:6 on irradiated MEFs. CHIR99021, DMH-1, SB431542, VPA, purmorphamine, and RA were added at same concentrations as described above. To induce differentiation into mature MNs, CHIR99021, DMH-1, and SB431542 were withdrawn from the medium, and MN progenitors were cultured in the basic neural medium (DMEM/F12, Neurobasal medium at 1:1, 1× N2 neural supplement, 1× B27 neural supplement, and 1 mM ascorbic acid) plus 0.1 μM RA and 0.1 μM purmorphamine for 1 week to generate MNX1⁺ post-mitotic MNs, and then differentiated into CHAT mature MNs in another 1-2 weeks.

We claim:

1. A method for maintaining a population of neuronal subtype-specific progenitors, the method comprising culturing neuronal subtype-specific progenitors in a culture medium comprising a Wnt signaling pathway agonist, an inhibitor of the bone morphogenetic protein (BMP) signaling pathway, an inhibitor of the transforming growth factor beta (TGFIβ) signaling pathway, and a Notch signaling pathway agonist, whereby expression of a neuronal subtype-specific progenitor gene expression profile is maintained in the neuronal subtype-specific progenitors.

2. The method of claim 1, wherein the neuronal subtype-specific progenitors have a gene expression profile comprising expression of at least one of SOX1, SOX2, NESTIN, N-Cadherin, and Ki67.

3. The method of claim 2, wherein the neuronal subtype-specific progenitors are spinal neural progenitors having a gene expression profile further comprising expression of at least one of HOXA5 and HOXB8, and substantially no expression of midbrain, hindbrain, or forebrain markers.

4. The method of claim 3, wherein the spinal neural progenitors are OLIG2⁺ spinal motor neuron progenitors.

5. The method of claim 2, wherein the neuronal subtype-specific progenitors are hindbrain neural progenitors having a gene expression profile further comprising expression of at least one of GBX2, KROX20, HOXA1-4, and HOXB1-4, and substantially no expression of forebrain, spinal cord, or midbrain markers.

6. The method of claim 5, wherein the hindbrain neural progenitors are NKX2.2⁺ hindbrain serotonergic neural progenitors.

7. The method of claim 2, wherein the neuronal subtype-specific progenitors are midbrain neural progenitors having a gene expression profile further comprising expression of at least one of EN1 and EN2, and substantially no expression of forebrain, spinal cord, or hindbrain markers.

8. The method of claim 7, wherein the midbrain neural progenitors are LMX1A⁺ midbrain dopaminergic neuron progenitors.

9. The method of claim 2, wherein the neuronal subtype-specific progenitors are forebrain neural progenitors having a gene expression profile further comprising expression of at least one of FOXG1 and OTX2, and substantially no expression of midbrain, spinal cord, or hindbrain markers.

10. The method of claim 9, wherein the forebrain neural progenitors are NKX-2.1⁺ forebrain GABAergic neuron progenitors.

11. The method of claim 1, wherein the Wnt signaling pathway agonist is a GSK3 inhibitor selected from the group consisting of CHIR99021 and 6-bromo-iridium-3′-oxime.

12. The method of claim 1, wherein the BMP signaling pathway inhibitor is selected from the group consisting of DMH-1, Dorsomorphin, and LDN-193189.

13. The method of claim 1, wherein the Notch signaling pathway agonist is a histone deacetylase (HDAC) inhibitor selected from the group consisting of valproic acid (VPA), suberoyl bis-hydroxamic acid (SBHA), and sodium butyrate.

14. The method of claim 1, wherein the TGFIβ signaling pathway inhibitor is selected from the group consisting of SB431542, SB505124, and A83-01.

15. The method of claim 1, wherein the culture medium comprises CHIR99021, DMH-1, SB431542, and VPA.

16. The method of claim 15, wherein the culture medium comprises between about 1 μM-3 μM CHIR99021; about 1 μM-5 μM DMH-1; about 1 μM-5 μM SB431542; and about 0.2-μM-2 μM VPA.

17. The method of claim 4, wherein the neuronal subtype specific progenitors are OLIG2⁺ spinal motor neuron progenitors, and wherein the culture medium comprises CHIR99021, DMH-1, SB431542, VPA, a SHH pathway agonist, and a RA pathway agonist.

18. The method of claim 17, wherein the SHH pathway agonist is selected from the group consisting of purmorphamine and SAG (Smoothened Agonist).

19. The method of claim 17, wherein the RA pathway agonist is retinoic acid.

20. The method of claim 17, wherein the culture medium comprises between about 1 μM to 3 μM CHIR99021; about 1 μM to 5 μM DMH-1; about 1 μM to 5 μM SB431542; about 0.2 μM-2 μM VPA; and about 0.1 μM to 1 μM purmorphamine; about 0.01 μM to 1 μM RA.

21. The method of claim 17, wherein the OLIG2⁺ spinal motor neuron progenitors are maintained in a culture substantially free of MNX1⁺ post-mitotic motor neurons for at least 5 weeks.

22. The method of claim 17, wherein the OLIG2⁺ spinal motor neuron progenitors are maintained in a culture substantially free of MNX1⁺ post-mitotic motor neurons for at least 10 weeks.

23. The method of claim 6, wherein the neuronal subtype specific progenitors are NKX2.2⁺ hindbrain serotonergic neural progenitors, and wherein the culture medium comprises CHIR99021, DMH-1, SB431542, VPA, and purmorphamine.

24. The method of claim 23, wherein the culture medium comprises about 1 μM to 3 μM CHIR99021; about 1 μM to 5 μM DMH-1; about 1 μM to 5 μM SB431542; about 0.2 μM-2 μM VPA; and about 0.1 μM to 1 μM purmorphamine

25. The method of claim 23, wherein the NKX2.2⁺ hindbrain serotonergic neural progenitors are maintained substantially free from differentiation for at least 5 weeks.

26. The method of claim 23, wherein the NKX2.2⁺ hindbrain serotonergic neural progenitors are maintained substantially free from differentiation for at least 10 weeks.

27. The method of claim 1, wherein the neuronal subtype specific progenitors are obtained from pluripotent stem cells.

28. The method of claim 27, wherein the pluripotent stem cells are human pluripotent stem cells.

29. The method of claim 28, wherein the human pluripotent stem cells are human embryonic stem cells.

30. The method of claim 28, wherein the human pluripotent stem cells are human induced pluripotent stem cells.

31. The method of claim 1, wherein the neuronal subtype specific progenitors are obtained from a human embryo.
